(19) United States
(12) Patent Application Publication
(10) Pub. No.: US 2010/0173779 A1
(43) Pub. Date: Jul. 8, 2010

(54) COMPOSITION AND METHOD TO STIMULATE GROWTH AND DEFENSE AGAINST PATHOGENS IN PLANTS

(76) Inventor: Alejandra Leonor Solange Moenne Munoz, Santiago (CL)

Correspondence Address: LADAS & PARRY LLP, 224 SOUTH MICHIGAN AVENUE, SUITE 1600, CHICAGO, IL, 60604, US

(21) Appl. No.: 12/666,700

(22) PCT Filed: Aug. 12, 2008
(86) PCT No.: PCT/IB08/02129

§ 371 Date: Dec. 24, 2009

(30) Foreign Application Priority Data
Aug. 16, 2007 (CL) 2369-2007

(51) Int. Cl. A01N 43/16; A61K 31/731; A01P 21/00; A61P 31/00

(52) U.S. Cl. 504/292; 514/54

(57) ABSTRACT

An aqueous composition to stimulate growth and defense against pathogens and other favorable productive properties in plants, including an oligo-carrageenan selected from kappa 1, kappa 2, lambda or iota oligo-carrageenans. Also, a method of spraying said the composition over the leaves of the plants and its use to stimulate defense against tobacco mosaic virus (TMV) and increase the height, the foliar biomass and the number of leaves in a plant. Also including a preparation method of the composition.

The present application relates to a composition and method to stimulate growth and defense against pathogens and other favorable productive properties in plants. Specifically, it relates to an aqueous composition of kappa1, kappa2, lambda or iota oligo-carrageenans, to the treatment of plants, and to their properties to stimulate growth and defense against pathogens. These oligo-carrageenans are polymers of around 20 units of galactose linked by alternate α-1,4 and β-1,3 bonds with sulphatations on carbons 2 or 4 of the A ring (first galactose) and/or on positions 2 or 6 of the B ring (second galactose). The aqueous composition of each of the four oligo-carrageenans is sprayed on plant leaves in order to stimulate growth, defense against pathogens and other productive properties.

### PREVIOUS ART

For over five years, efforts have been made to prepare depolymerized fractions from polysaccharides extracted from Chilean marine algae showing favorable biological activities in plants, such as the stimulation of growth and the defense against pathogens. Initially, the effect of three fractions of oligosaccharides prepared via chemical hydrolysis from Chilean algae polysacharides were studied on the stimulation of growth and defense against tobacco mosaic virus (TMV) in tobacco plants (Laporte D., Vera, J., Chandía, N. P., Zúñga, E. A., Matsuhiro, B. y Moenne A., Structurally unrelated oligosaccharides differentially stimulate growth and defense against tobacco mosaic virus in tobacco plants, Journal of Applied Phycology 19, 79-88, 2007). The first fraction tested pertains to an oligosaccharide (Poly-Gu), made of around 20 units of guluronic acid, prepared via acid hydrolysis of alginates obtained from the brown seaweed Lessonia trabeculata. The second fraction pertains to an oligosaccharide (Poli-Ma), made of around 20 units of mannuronic acid, prepared via acid hydrolysis of alginates obtained from the brown seaweed Lessonia vadosa. The third fraction pertains to an oligosaccharide (Poli-Ga), made of around 20 units of sulphated galactose with sulphatations on positions 2, 3 and 4 of the galactose ring, prepared via free radical hydrolysis of the sulphated galactan polymer obtained from the red seaweed Schyzimenia binderi. Spraying of an aqueous solution of each of these oligosaccharides on tobacco leaves at a concentration of 0.5 mg/mL differentially stimulates growth and defense against TMV in tobacco plants (Xhanti NN). Height and biomass of tobacco plants was particularly stimulated by Poli-Ga and Poli-Ma oligosaccharides, approximately 23% and 49% respectively, as compared with the controls sprayed with water. On the contrary, the Poli-Gu showed no effect on growth. On the other hand, Poli-Ga, Poli-Ma and Poli-Gu oligosaccharides stimulate defense against TMV infection by 74%, 22% and 9%, when analyzed 15 days after treatment. Therefore, Poli-Ga oligosaccharide induces a slight stimulation of growth and a strong stimulation of defense against TMV in tobacco plants. These efforts gave rise to a patent filed on March 2005 at the Chilean Industrial Property Office with Privilege Number 0693-2005, currently under revision.

As Poli-Ga oligosaccharide is difficult to obtain because the red seaweed Schyzimenia binderi is not abundant in the Chilean coast, the option of replacing Poli-Ga by oligo-carrageenans obtained from commercial carragenans (Gelymar S. A.) was revised. These carrageenans are extracted from the red carrageenophytes seaweeds Sarcothalia crispata and Gigartina skotsbergii. Kappa 1, kappa 2, lambda and iota commercial carrageenans are galactose polymers attached by alternate bonds α-1,4 y β-1,3 with sulphatations on carbon 2 or 4 of the A ring (first galactose) and/or on position 2 or 6 of the B ring (second galactose). The mentioned commercial carrageenans were solubilized in hot water and depolymerized by acid hydrolysis resulting in four fractions of around 20 units of sulphated galactose corresponding to the kappa1, kappa2, lambda and iota carrageenans. These oligo-carrageenans were sprayed on the tobacco leaves at a concentration of 0.5, 1 and 5 mg/L and they induced a strong stimulation of growth and defense against TMV, when analyzed 45 days after treatment. Particularly, kappa1 and kappa2 oligo-carrageenans at a concentration of 0.5 mg/mL induced a significant increase of foliar biomass (between 133 and 175%) and lambda and iota oligo-carrageenans at a concentration of 1-5 mg/mL strongly protect tobacco plants (Xhanti NN) against TMV (80-95% protection). It should be noted that the effect of each oligo-carrageenan is different from that of Poli-Ga when assayed in tobacco plants, specially considering that all of them have a similar molecular weight, that the same concentration of each was used, and that the effect was analyzed in the same cultivar of tobacco plants. This proves that oligo-carrageenans are agents that stimulate growth and defense against pathogens in plants different from Poli-Ga. Moreover, although the four oligo-carrageenans are attached by α-1,4 y β-1,3 bonds and they differ only in sulphatation positions, their effect on growth and defense against pathogens in tobacco plants is also different, which proves that these oligo-carrageenans are essentially different agents from each other.

### BRIEF DESCRIPTION OF THE FIGURES

FIG. 1A: Effect of distinct concentrations of kappa1 oligo-carrageenan on the height of tobacco plants (Xhanti NN).

FIG. 1B: Effect of distinct concentrations of kappa1 oligo-carrageenan on the foliar biomass of tobacco plants (Xhanti NN).

FIG. 1C: Effect of distinct concentrations of kappa1 oligo-carrageenan on the number of necrotic lesions produced by tobacco mosaic virus infection in tobacco plants (Xhanti NN).

FIG. 2A: Effect of distinct concentrations of kappa2 oligo-carrageenan on the height of tobacco plants (Xhanti NN).

FIG. 2B: Effect of distinct concentrations of kappa2 oligo-carrageenan on the foliar biomass of tobacco plants (Xhanti NN).

FIG. 2C: Effect of distinct concentrations of kappa2 oligo-carrageenan on the number of necrotic lesions produced by tobacco mosaic virus infection in tobacco plants (Xhanti NN).

FIG. 3A: Effect of the distinct concentrations of iota oligo-carrageenan on the height of tobacco plants (Xhanti NN).

FIG. 3B: Effect of the distinct concentrations of iota oligo-carrageenan on the foliar biomass of tobacco plants (Xhanti NN).

FIG. 3C: Effect of distinct concentrations of iota oligo-carrageenan on the number of necrotic lesions produced by tobacco mosaic virus infection in tobacco plants (Xhanti NN).

FIG. 4A: Effect of distinct concentrations of lambda oligo-carrageenan on the height of tobacco plants (Xhanti NN).

FIG. 4B: Effect of distinct concentrations of lambda oligo-carrageenan on the biomass of tobacco plants (Xhanti NN).

FIG. 4C: Effect of distinct concentrations of lambda oligo-carrageenan on the number of necrotic lesions produced by tobacco mosaic virus infection in tobacco plants (Xhanti NN).

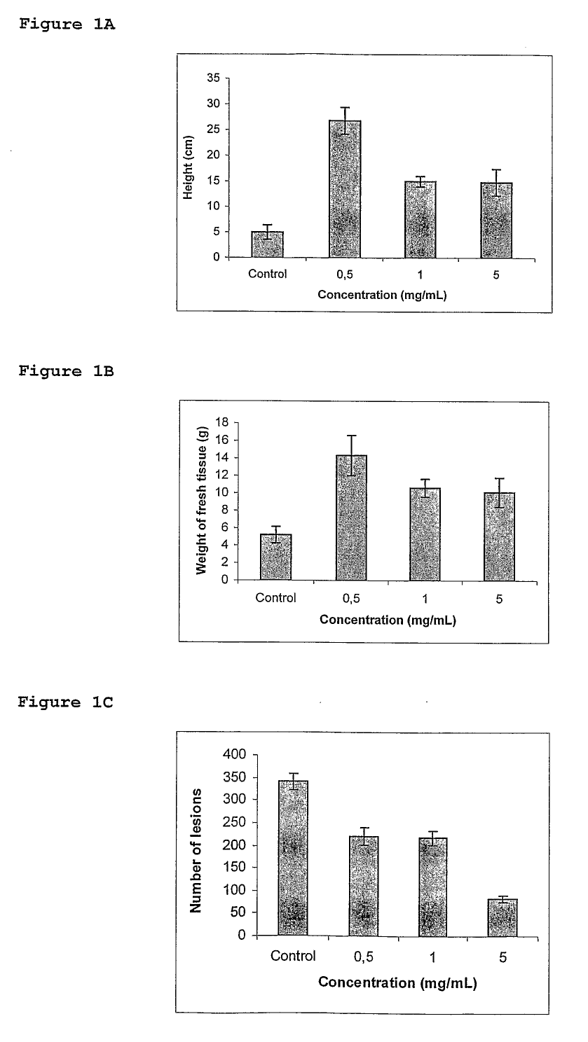

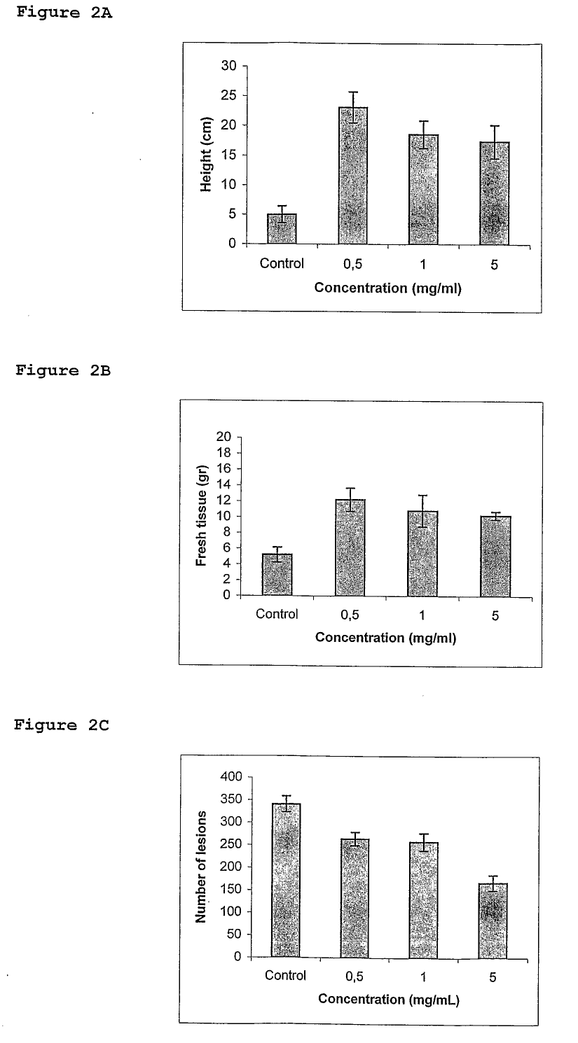

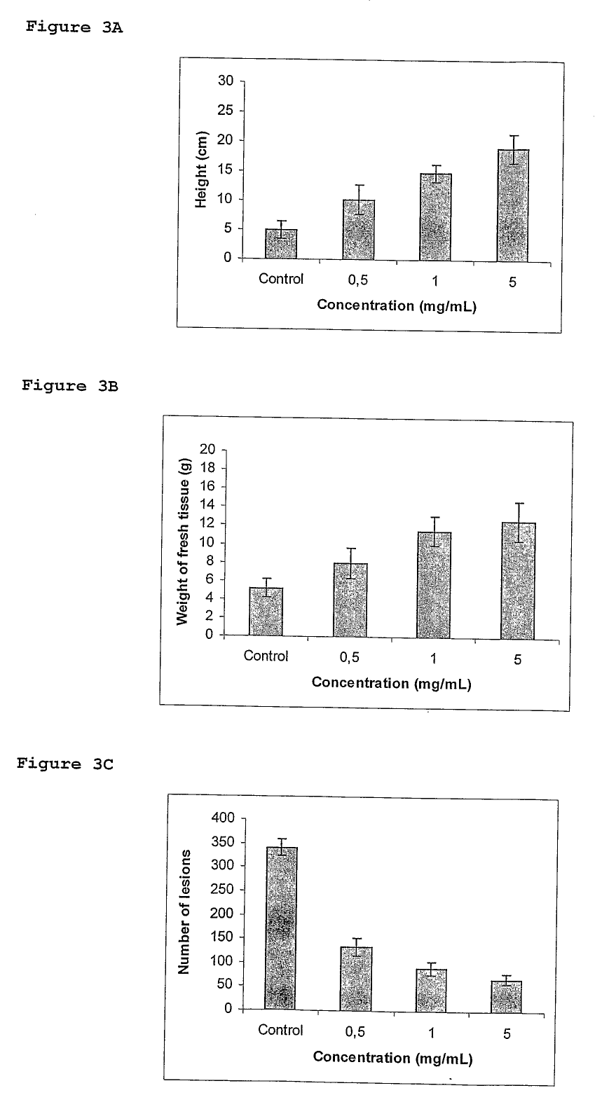

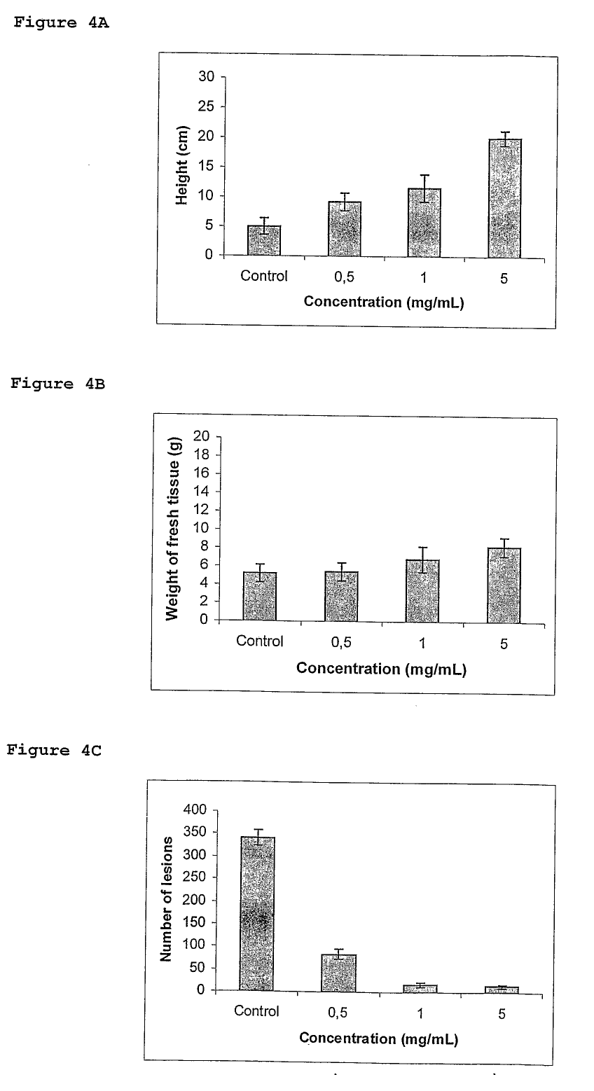

### DETAILED DESCRIPTION OF THE INVENTION

Carrageenans kappa1, kappa2, iota and lambda are polymers of sulphated galactose units linked by alternate α-1,4 and β-1,3 bonds with sulphatations in distinct positions of the galactose A and B rings (first and second galactose). Kappa1 carrageenan has a sulphatation on carbon 4 of the A ring and an anhydrous bond between carbons 3 and 6 of the B ring. Kappa2 carrageenan has a sulphatation on carbon 4 of the A ring, a high percentage of sulphatation (40 to 60%) on carbon 2 of the B ring and an anhydrous bond between carbons 3 and 6 of the B ring. Iota carrageenan has a sulphatation on carbon 4 of the A ring, a sulphatation on position 2 of the B ring and an anhydrous bond between carbons 3 and 6 of the B ring. The lambda carrageenan has a high percentage of sulphatation on carbon 2 of A ring (70%) and a sulphatation on carbon 2 and 6 of the B ring (this carrageenan has no anhydrous bond in the B ring).

The depolymerization of kappa1, kappa2, iota and lambda carrageenans was made by acid hydrolysis adding 4N hydrochloric acid to an aqueous solution of the carrageenan at a concentration of 10 mg/mL concentration hot-incubated. In order to avoid significant alteration of the degree of sulphatation in the depolymerized products, the time of hydrolysis was short. The depolymerization reaction was stopped by adding 4N sodium hydroxide until a pH equal to 7.0 was obtained. The depolymerization products (oligo-carrageenans) were analyzed by electrophoresis in a 2% agarose gel and stained with 1% Alciane Blue dye which binds to sulphated sugars. In every case, products of uniform sizes were obtained with an approximate weight of 8,500 Daltons (no family of different size products was obtained, as could have been expected). This shows that the depolymerization products (oligo-carrageenans) pertain to oligosaccharides of approximately 20 units of sulphated galactose. The oligo-carrageenans were concentrated using dialysis bags and sucrose outside to extract water. Then, the semi-solid product was taken to dryness in an oven at 40° C. The solid product was pulverized using a mortar and easily solubilized in cold distilled water.

Oligo-carrageenans kappa1, kappa2, iota and lambda were solubilized in water at a concentration of 0.5, 1 and 5 mg/mL and they were sprayed (1 mL) on the upper and lower side of leaves of Xhanti NN tobacco plants (starting height being 3 cm). A group of 10 tobacco plants (treatment group) was sprayed with an oligo-carrageenan at a certain concentration and a group of 10 plants was sprayed only with water (control group). The sprayings were run once a week, three times in total, and then tobacco plants were grown for 45 days at ambient temperature (summer). The height of each plant in the control group (n=10) and that of each plant in the treated groups (n=10) was determined as well as the number of leaves (n=10) and foliar biomass (n=5). In addition, one leave of each plant (n=5) of the control group and of the treatment group was infected using abrasive carburundum and 100 μL of a tobacco mosaic virus (TMV) suspension at a protein concentration of 0.4 mg/mL. After five days, the number of necrotic lesions was counted in the control and treated groups (TMV infection produces necrotic lesions in Xhanti NN tobacco plants) and the percentage of decrease in the number of necrotic lesions (percentage of protection) was determined.

The results of these experiments showed that the four oligo-carrageenans stimulate the growth in height of tobacco plants, mainly kappa1 and kappa2 oligo-carrageenans at a concentration of 0.5 mg/mL, which induced an increase in height of 436% and 362%, respectively. Likewise, the four oligo-carrageenans increase the weight of foliar biomass, mainly kappa1 and kappa2 oligo-carrageenans at a concentration of 0.5 mg/mL and iota oligo-carrageenan at a concentration of 5 mg/mL, which increased the weight by 175%, 135% and 142%, respectively. Moreover, the four oligo-carrageenans increase the number of leaves, mainly kappa1 oligo-carrageenan at a concentration of 0.5 mg/mL and lambda oligo-carrageenan at a concentration of 5 mg/mL, which increased the number of leaves by 100% and 80%, respectively. Finally, the four oligo-carrageenans induce protection against TMV infection, mainly lambda oligo-carrageenan at a concentration of 1 mg/mL and iota oligo-carrageenan at a concentration of 5 mg/mL, which increased protection by 96% and 80%, respectively. Therefore, the four oligo-carrageenans stimulate growth of tobacco plants, particularly kappa1 and kappa2 oligo-carrageenans, and defense against virus, particularly lambda and iota oligo-carrageenans.

The following tables show results obtained with kappa1, kappa2, iota and lambda oligo-carrageenans used in stimulating growth and defense in Xhanti NN tobacco plants.

TABLE I
Height of Xhanti NN tobacco plants treated with
distinct concentrations of kappa1, kappa2, iota and lambda
oligo-carrageenans and the percentage of difference in height.

| | | Percentage of |
|---|---|---|
| | Plant height | difference in height |
| | (cm) | (%) |
| Carrageenan κ1 | | |
| Control | 5.0 ± 1.5 | 0 |
| 0.5 mg/mL | 26.8 ± 2.6 | 436 |
| 1 mg/mL | 15.0 ± 1 | 200 |
| 5 mg/mL | 14.8 ± 2.6 | 196 |
| Carrageenan κ2 | | |
| Control | 5.0 ± 1.5 | 0 |
| 0.5 mg/mL | 23.1 ± 2.6 | 362 |
| 1 mg/mL | 18.6 ± 2.3 | 272 |
| 5 mg/mL | 17.4 ± 2.8 | 248 |
| Carrageenan ι | | |
| Control | 5.0 ± 1.5 | 0 |
| 0.5 mg/mL | 10.2 ± 2.5 | 104 |
| 1 mg/mL | 14.8 ± 1.5 | 196 |
| 5 mg/mL | 19.0 ± 2.4 | 280 |
| Carrageenan λ | | |
| Control | 5.0 ± 1.5 | 0 |
| 0.5 mg/mL | 9.2 ± 1.5 | 84 |
| 1 mg/mL | 11.6 ± 2.3 | 132 |
| 5 mg/mL | 20.0 ± 1.2 | 300 |

Leaves of a group of 10 Xhanti NN tobacco plants (n=10) were sprayed with 1 mL of aqueous solution of kappa1, kappa2, iota and lambda oligo-carrageenans at a concentration of 0.5, 1 and mg/mL and a control group of 10 plants (n=10) was sprayed only with water. The height (cm) of each plant in the control group and in the treated groups was determined and the average value and standard deviation were obtained in each case. The percentage of difference in height was calculated subtracting the average height of the control group from the average height of each treated group and considering the height of the control group as 100%.

TABLE II
Foliar biomass of tobacco plants (Xhanti NN) treated
with distinct concentrations of oligo-carrageenans and the
percentage of difference in foliar biomass.

| | | Percentage of |
|---|---|---|
| | Foliar biomass | difference in foliar |
| | (g) | biomass |
| Carrageenan κ1 | | |
| Control | 5.2 ± 1 | 0 |
| 0.5 mg/mL | 14.3 ± | 175 |
| 1 mg/mL | 10.6 ± | 104 |
| 5 mg/mL | 10.1 ± | 94 |
| Carrageenan κ2 | | |
| Control | 5.2 ± 1 | 0 |
| 0.5 mg/mL | 12.1 ± 1.5 | 133 |
| 1 mg/mL | 10.8 ± 2 | 108 |
| 5 mg/mL | 10.2 ± 0.5 | 96 |
| Carrageenan ι | | |
| Control | 5.2 ± 1 | 0 |
| 0.5 mg/mL | 8.0 ± 1.6 | 94 |
| 1 mg/mL | 11.5 ± 1.6 | 121 |
| 5 mg/mL | 12.6 ± 2.1 | 142 |
| Carrageenan λ | | |
| Control | 5.2 ± 1 | 0 |
| 0.5 mg/mL | 5.4 ± 1 | 4 |
| 1 mg/mL | 6.8 ± 1.4 | 31 |
| 5 mg/mL | 8.2 ± 1.9 | 58 |

Leaves of a group of 10 Xhanti NN tobacco plants (n=10) were sprayed with 1 mL of aqueous solution of kappa1, kappa2, iota and lambda oligo-carrageenans at a concentration of 0.5, 1 and 5 mg/mL and a control group of 10 plants (n=10) was sprayed only with water. The weight of leaves (g of fresh tissue) of each plant in the control group and in the treated groups was determined and the average value and the standard deviation were obtained in each case. The percentage of difference in foliar biomass was calculated by subtracting the average biomass of the control group from the average biomass of each treated group and considering the weight of the control group as 100%.

TABLE III
Number of leaves of Xhanti tobacco plants NN treated
with different concentrations of oligo-carrageenans and the
percentage of difference in the number of leaves.

| | | Percentage of |
|---|---|---|
| | | difference in the |
| | Number of leaves | number of leaves |
| Carrageenan κ1 | | |
| Control | 5 ± 1 | 0 |
| 0.5 mg/mL | 10 ± 1 | 100 |
| 1 mg/mL | 7 ± 1 | 40 |
| 5 mg/mL | 7 ± 1 | 40 |
| Carrageenan κ2 | | |
| Control | 5 ± 1 | 0 |
| 0.5 mg/mL | 7 ± 1 | 40 |
| 1 mg/mL | 6 ± 1 | 20 |
| 5 mg/mL | 6 ± 1 | 20 |
| Carrageenan ι | | |
| Control | 5 ± 1 | 0 |
| 0.5 mg/mL | 6 ± 2 | 20 |
| 1 mg/mL | 7 ± 1 | 40 |
| 5 mg/mL | 7 ± 2 | 40 |
| Carrageenan λ | | |
| Control | 5 ± 1 | 0 |
| 0.5 mg/mL | 5 ± 1 | 0 |
| 1 mg/mL | 6 ± 1 | 20 |
| 5 mg/mL | 9 ± 2 | 80 |

Leaves of a group of 10 Xhanti NN tobacco plants leaves (n=10) were sprayed with 1 mL of aqueous solution of kappa1, kappa2, iota and lambda oligo-carrageenans at a concentration of 0.5, 1 and 5 mg/mL and the control group of 10 plants (n=10) was sprayed only with water. The number of leaves of each plant in the control group and in the treated groups was determined and the average value and the standard deviation were calculated in each case. The percentage of difference in the number of leaves was calculated by subtracting the average value in the control group from the average value of each treated group, and considering the number of leaves in the control group as 100%.

TABLE IV
Number of necrotic lesions in Xhanti NN tobacco plants
treated with distinct concentrations of oligo-carrageenans and
infected with tobacco mosaic virus and the percentage of
protection against viral infection.

| | | Percentage of |
|---|---|---|
| | Number of necrotic | protection against |
| | lesions per leaf | TMV |
| Carrageenan κ1 | | |
| Control | 341 ± 18 | 0 |
| 0.5 mg/mL | 221 ± 19 | 35 |
| 1 mg/mL | 217 ± 15 | 36 |
| 5 mg/mL | 83 ± 16 | 76 |
| Carrageenan κ2 | | |
| Control | 341 ± 18 | 0 |
| 0.5 mg/mL | 264 ± 15 | 23 |
| 1 mg/mL | 258 ± 19 | 24 |
| 5 mg/mL | 167 ± 17 | 51 |
| Carrageenan ι | | |
| Control | 341 ± 18 | 0 |
| 0.5 mg/mL | 134 ± 19 | 61 |
| 1 mg/mL | 90 ± 15 | 74 |
| 5 mg/mL | 68 ± 10 | 80 |
| Carrageenan λ | | |
| Control | 341 ± 18 | 0 |
| 0.5 mg/mL | 85 ± 11 | 75 |
| 1 mg/mL | 17 ± 5 | 95 |
| 5 mg/mL | 14 ± 4 | 96 |

Leaves of a group of 10 Xhanti NN tobacco plants (n=10) were sprayed with 1 mL of aqueous solution of kappa1, kappa2, iota and lambda oligo-carrageenans at a concentration of 0.5, 1 and mg/mL and a control group of 10 plants (n=10) was sprayed only with water. The number of necrotic lesions in each plant of the control group and in the treated groups was determined and the average value and the standard deviation were calculated in each case. The percentage of protection against the TMV infection was calculated by subtracting the average value of necrotic lesions in each treated group from the average value of necrotic lesions in the control group and this difference turned into a percentage considering the control group as 100% infection or 0% protection.

1. An aqueous composition to stimulate growth and defense against pathogens and other favorable productive properties in plants, comprising an oligo-carrageenan selected from kappa1, kappa2, lambda or iota oligo-carrageenans having 20 units of sulphated galactose linked by alternate α-1, 4 and β-1, 3 bonds with sulphtations on carbons 2 or 4 of the A ring (first galactose) and/or on positions 2 or 6 of the B ring (second galactose).

2. The aqueous composition of claim 1, comprising 0.5, 1 or 5 mg/mL of an oligo-carrageenan selected from kappa1, kappa2, lambda or iota oligo-carrageenans.

3. The aqueous composition of claim 2, comprising 0.5 mg/mL of an oligo-carrageenan selected from kappa1 or kappa2 oligo-carrageenans.

4. The aqueous composition of claim 2, comprising 5 mg/mL of an oligo-carrageenan selected from iota oligo-carrageenan.

5. The aqueous composition of claim 2, comprising 1 mg/mL of an oligo-carrageenan selected from lambda oligo-carrageenan.

6. A method to stimulate growth and defense against pathogens and other favorable productive properties in plants, comprising the application of the aqueous composition of claim 1 by spraying plant leaves.

7. The method of claim 6, characterized in that it comprises spraying the aqueous composition of on the upper and lower parts of plant leaves.

8. The method of claim 6, wherein the plant is a tobacco plant.

9. The method of claim 8, further comprising spraying once a week, at least three times, and growing of the plant is continued.

10. The method of claim 9, wherein the plants are grown at least for the next 45 days after treatment.

11. The method of claim 10, wherein the plants are grown at ambient temperature.

12. The use of the composition of claim 1 to stimulate growth and defense against pathogens in plants.

13. The use of claim 12 to stimulate the defense against TMV in plants.

14. The use of claim 12 to stimulate defense against TMV in tobacco plants.

15. The use of claim 12 to increase the height, the foliar biomass and the number of leaves in a plant.

16. The use of claim 15, to increase the height, the foliar biomass and the number of leaves in a tobacco plant.

17. The use of claim 12, wherein the aqueous composition comprises 0.5 mg/mL of kappa1 or kappa2 oligo-carrageenans.

18. The use of claim 12, wherein the aqueous composition comprises 0.5 mg/mL kappa1 oligo-carrageenan.

19. The use of claim 12, wherein the aqueous composition comprises 5 mg/mL lambda oligo-carrageenan.

20. The use of claim 12 wherein the aqueous composition comprises 1 or 5 mg/mL lambda or iota oligo-carrageenans.

21. The use of claim 20 to increase the defense against TMV infection in tobacco plants.

22. (canceled)

23. Preparation method of the aqueous composition of claim 1, comprising:
using carrageenans obtained from Chilean carrageenophyte seaweeds Sarcothalia crispata and Gigartina skotsbergll;
solubilizing the carrageenans in hot water; and
depolymerizing the carrageenans by acid hydrolysis to obtain fractions of approximately 20 units of sulphated galactose in distinct positions of the galactose ring corresponding to kappa1, kappa2, lambda and iota oligo-carrageenans.

24. The method of claim 23, further comprising performing the depolymerization of kappa1, kappa2, iota and lambda oligo-carrageenans by hot acid hydrolysis adding hydrochloric acid to an aqueous solution of carrageenans at a concentration of 10 mg/mL, such as not to significantly alter the degree of sulphatation of the depolymerized products; and stop the depolymerization reaction by adding sodium hydroxide to obtain a pH equal to 7.0.

25. The method of claim 24 comprising concentrating the oligo-carrageenans using dialysis bags and sucrose outside to extract the water; drying the semisolid product to dryness in an oven at 40° C.; pulverizing the solid product in a mortar; and solubilizing it in cold distilled water.
